(19) United States
(12) Patent Application Publication
(10) Pub. No.: US 2021/0154119 A1
(43) Pub. Date: May 27, 2021

(54) ASMT EXPRESSION PROMOTER

(71) Applicant: Shiseido Company, Ltd., Chuo-ku, Tokyo (JP)

(72) Inventors: Yoko GOZU, Kanagawa (JP); Shinichiro HAZE, Kanagawa (JP)

(73) Assignee: Shiseido Company, Ltd., Chuo-ku, Tokyo (JP)

(21) Appl. No.: 17/044,920

(22) PCT Filed: Apr. 5, 2018
(86) PCT No.: PCT/JP2018/014614

§ 371 Date: Oct. 2, 2020

(51) Int. Cl. A61K 8/49; A61Q 19/00; A23L 33/10

(52) U.S. Cl. CPC A61K 8/4953; A23L 33/10; A61Q 19/00

(57) ABSTRACT

The objective of the present invention is to provide a novel ASMT expression promoter. Provided are an ASMT expression promoter comprising ectoine or a physiologically acceptable salt thereof as an active ingredient, and a melatonin synthesis promoter comprising ectoine or a physiologically acceptable salt thereof as an active ingredient, which promotes melatonin synthesis through the promotion of ASMT expression. Effects such as improvement of sleep disorder, improvement of cognitive function, improvement of mood disorder, enhancement of antioxidant activity, and enhancement of anti-inflammatory activity can be enhanced by promoting melatonin synthesis through the promotion of ASMT expression.

### TECHNICAL FIELD

The present invention relates to an ASMT expression promoter.

### BACKGROUND ART

Melatonin is a hormone present in plants and animals and has been reported to have effects such as improvement of sleep disorders, improvement of cognitive function, improvement of mood disorders, and enhancement of antioxidant activity/anti-inflammatory activity (PTL 1 and 2, NPL 1).

Acetyl serotonin O-methyl transferase (ASMT) is known as a substance to promote melatonin synthesis. ASMT is an enzyme produced in the body by the expression of ASMT gene and is involved in the final step of the melatonin synthesis pathway. Therefore, it is considered that the promotion of ASMT expression would enhance melatonin synthesis, thereby increasing the amount of melatonin (PTL 2, and NPL 1 to 6).

Therefore, it is desirable to search for substances that promote melatonin synthesis by promoting ASMT expression. However, it has not been sufficiently elucidated so far as to which substance can greatly contribute to the ASMT expression promoting action.

### CITATION LIST

### Patent Literature

[PTL 1] Japanese Unexamined Patent Publication (Kokai) No. 2014-237700
[PTL 2] Japanese Unexamined Patent Publication (Kohyo) No. 2009-511038
[PTL 3] Japanese Unexamined Patent Publication (Kokai) No. 2002-302444
[PTL 4] Japanese Patent No. 5116917

### Non-Patent Literature

[NPL 1] Atsuhiko Hattori, Review Melatonin and Aging, Comparative Physiology and Biochemistry Vol. 34 (2017), No. 1, p. 2-11
[NPL 2] Pagan et.al., Mutation screening of ASMT, the last enzyme of the melatonin pathway, in a large sample of patients with Intellectual Disability, BMC Medical Genetics 2011, 12:17
[NPL 3] Reiter et.al., Melatonin and its metabolites: new findings regarding their production and their radical scavenging actions, Acta Biochimica Polonica, Vol. 54 No. 1, 2007, 1-9
[NPL 4] Ribelayga et.al., HIOMT drives the photoperiodic changes in the amplitude of the melatonin peak of the Siberian hamster, American Journal of Physiology Regulatory Integrative Comparative Physiology, 278: R1339-R1345, 2000.
[NPL 5] Ceinos et.al., Analysis of Adrenergic Regulation of Melatonin Synthesis in Siberian Hamster Pineal Emphasizes the Role of HIOMT, Neurosignals 2004; 13: 308-317
[NPL 6] Monika Talarowska et. al., ASMT gene expression correlates with cognitive impairment in patients with recurrent depressive disorder, Medical Science Monitor, 2014; 20: 905-912
[NPL 7] Yi H1 et. al., Localization of the hydroxyindole-O-methyltransferase gene to the pseudoautosomal region: implications for mapping of psychiatric disorders, Human Molecular Genetics. 1993 February; 2(2): 127-31.
[NPL 8] Gaia Favero et. al., Melatonin as an Anti-Inflammatory Agent Modulating Inflammasome Activation, International Journal of Endocrinology Volume 2017, Article ID 1835195, 13 pages.
[NPL 9] Andrze Alominski, D. J. Tobin, M. A. Zmijewski, et.al., Melatonin in the skin: synthesis, metabolism and functions. Tremds in Endocrinology & Metabolism, 2008, 19: 17-24
[NPL 10] D X Tan, L D Chen, B Poeggeler, et.al., Melatonin: a potent endogenous hydroxyl radical scavenger. Endocrine J, 1993, 1: 57-60

### SUMMARY OF INVENTION

### Technical Problem

This is an objective of the present invention to provide a novel ASMT expression promoter.

### Solution to Problem

As a result of intensive research on the effects of various components as an ASMT expression promoter, the inventors have found that ectoine has a particularly high effect as an ASMT expression promoter, and have completed the following inventions:

(1) An ASMT expression promoter comprising ectoine or a physiologically acceptable salt thereof as an active ingredient.
(2) A melatonin synthesis promoter comprising ectoine or a physiologically acceptable salt thereof as an active ingredient, which promotes melatonin synthesis through the promotion of ASMT expression.
(3) An antioxidant comprising ectoine or a physiologically acceptable salt thereof as an active ingredient, which enhances antioxidant activity by promoting melatonin synthesis through the promotion of ASMT expression.
(4) A composition comprising the ASMT expression promoter according to (1).
(5) A composition comprising the melatonin synthesis promoter according to (2).
(6) A composition comprising the antioxidant according to (3).
   (7) The composition according to (4) or (5) for enhancing one or more effects selected from improvement of sleep disorder, improvement of cognitive function, improvement of mood disorder, enhancement of antioxidant activity, and enhancement of anti-inflammatory activity, by promoting melatonin synthesis through the promotion of ASMT expression.
   (8) The composition according to any one of (4) to (7), which is a cosmetic composition or a food composition.

### Advantageous Effects of Invention

ASMT expression can be promoted by the administration of the ASMT expression promoter of the present invention. According to the present invention, a composition comprising the ASMT expression promoter can be provided. Through the promotion of ASMT expression, melatonin synthesis can be promoted, thereby enhancing the effects of melatonin such as improvement of sleep disorder, improvement of cognitive function, improvement of mood disorder, enhancement of antioxidant activity, and enhancement of anti-inflammatory activity.

### BRIEF DESCRIPTION OF DRAWINGS

FIG. 1 shows the results of the ASMT expression promoting effect of ectoine as compared with a control containing no ectoine. The results are shown as relative values as a comparison with the control the ASMT gene expression level of which is assumed to be 1.

FIG. 2 shows the results of the ASMT expression promoting effect of respective concentrations of ectoin. As an internal standard, the expression level of the RPLP0 gene, which is a housekeeping gene whose expression level is almost constant, was quantified, and the relative expression level of ASMT with respect to RPLP0, ASMT/RPLP0 was calculated as the ASMT gene expression level.

FIG. 3 shows the results of the ASMT expression promoting effect of ectoin as compared with various control drugs and a control without the addition of any antioxidant. As an internal standard, the expression level of the RPLP0 gene, which is a housekeeping gene whose expression level is almost constant, was quantified, and the relative expression level of ASMT with respect to RPLP0, ASMT/RPLP0 was calculated as the ASMT gene expression level.

### DESCRIPTION OF EMBODIMENTS

The present invention provides an ASMT expression promoter comprising ectoine or a physiologically acceptable salt thereof as an active ingredient.

Ectoine refers to (4S)-2-methyl-1,4,5,6-tetrahydropyrimidine-4-carboxylic acid, which is a cyclic amino acid having the following structure (CAS number 96702-03-3).

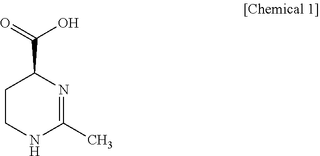

Ectoin is known to have an osmoregulating action, and it works to protect cells from stresses due to osmotic changes. In addition, ectoin is known to have an action to retain the skin moisture for a long time, and thus is contained in various types of cosmetics (PTL 3 and 4). However, it has not been reported so far that ectoin has a high ASMT expression promoting action, which is a very surprising finding.

Physiologically acceptable salts include alkali or alkaline earth salts, especially salts of potassium, sodium, magnesium, and calcium, as well as salts derived from organic bases such as non-toxic aliphatic or aromatic amines.

ASMT refers to acetyl serotonin O-methyl transferase (CAS number 9029-77-0). ASMT is known as an enzyme that catalyzes the reaction to convert normelatonin to melatonin at the final step in the melatonin synthesis pathway. Further, ASMT may also be called by other names such as HIOMT (hydroxyindole-O-methyltransferase) (NPL 1 to 5).

The ASMT gene is a gene that is located in the pseudoautosomal region 1 (PAR1) common to the X chromosome and the Y chromosome, and encodes acetyl serotonin O-methyl transferase (ASMT)(NPL 7). It has been suggested that a lower level of the ASMT gene expression would result in a decrease in the melatonin production amount because normelatonin cannot be converted to melatonin, and conversely, a higher level of the ASMT gene expression would result in an increase in the melatonin production amount (NPL 1 to 5).

Melatonin refers to N-acetyl-5-methoxytryptamine (CAS number 73-31-4). Melatonin is produced in the pineal gland in animals such as human, and in recent years it is also reported that melatonin is produced in other various organs such as skin, retina, brain, spinal cord, digestive tract, heart, kidney, testis, ovary, thymus, spleen, lens, cochlea, bone marrow, lymphocytel, etc. (NPL 1, 9, and 10). In addition, melatonin has been reported to have various actions such as regulation of circadian rhythm, improvement of sleep disorder, improvement of cognitive function, improvement of mood disorder, antioxidant action and anti-inflammatory action in organs such as skin. (PTL 1 and 2, NPL 1, 2, 6 to 10). Therefore, it is expected that if melatonin can be promoted by ectoin which is discovered by the present inventors to have a high ASMT expression promoting action, the above-mentioned various effects of melatonin will be enhanced.

The promotion of ASMT expression refers to for example, an enhanced expression level of the ASMT gene with a statistically significant difference, for example, at a significance level of 5% (according to Student's t-test, for example), in the case where the ASMT expression promoter is added, as compared to the case where the ASMT expression promoter is not added (control). Alternatively, the promotion of ASMT expression may refer to for example an enhanced expression level of the ASMT gene by for example, 10% or more, 20% or more, 30% or more, 40% or more, 50% or more, 60% or more, 70% or more, 80% or more, 90% or more, 100% or more, 200% or more, 300% or more, 400% or more, or 500% or more, in the case where the ASMT expression promoter is added, as compared to the case where the ASMT expression promoter is not added (control). The expression level of the ASMT gene can be determined by any known technique, for example, the method described in NPL 6.

Therefore, the present invention provides a melatonin synthesis promoter comprising ectoine or a physiologically acceptable salt thereof as an active ingredient, which promotes melatonin synthesis through the promotion of ASMT expression, as well as an antioxidant comprising ectoine or a physiologically acceptable salt thereof as an active ingredient, which enhances antioxidant activity by promoting melatonin synthesis through the promotion of ASMT expression. The antioxidant activity may be of the skin or such an organ. The present invention also provides a composition comprising an ASMT expression promoter, a melatonin synthesis promoter, or an antioxidant containing ectoin or a physiologically acceptable salt thereof as an active ingredient. The composition of the present invention may be a cosmetic composition or a food composition. Further, the composition of the present invention may be a composition for enhancing one or more effects selected from improvement of sleep disorder, improvement of cognitive function, improvement of mood disorder, enhancement of antioxidant activity, and enhancement of anti-inflammatory activity, by promoting melatonin synthesis through the promotion of ASMT expression.

The ASMT expression promoter, the melatonin synthesis promoter, the antioxidant, or the composition of the present invention comprise ectoine or a physiologically acceptable salt thereof as an active ingredient, for example, at 10 mass % or more, 20 mass % or more, 30 mass % or more, 40 mass % or more, 50 mass % or more, 60 mass % or more, 70 mass % or more, 80 mass % or more, 90 mass % or more, 95 mass % or more, or 99 mass % or more. In one embodiment, the ASMT expression promoter or the melatonin synthesis promoter or the antioxidant of the present invention may consist of ectoin or a physiologically acceptable salt thereof. Ectoin or a physiologically acceptable salt thereof used in the present invention can be obtained by any method, and is not limited to the production method, the supply source, etc.

The ASMT expression promoter, the melatonin synthesis promoter, the antioxidant, or the composition of the present invention can be administered externally or orally. The form of external administration can be arbitrarily selected fro, for example, cream, emulsion, liquid, sheet, spray, gel and the like. The form of oral administration can also be arbitrarily selected from, for example, tablet, supplement, beverage, powder and the like.

The cosmetic composition of the present invention may be any of various cosmetics including milky lotion, cream, beauty serum, lotion, pack, face wash, solid soap, body soap, shampoo, etc., which may take any form such as liquid, emulsion, cream, solid, sheet, spray, gel, foam, powder and the like. Moreover, the food composition of the present invention may be any of powder, beverage, or tablet, which may take any form such as powder, liquid, solid, granule, pellet, paste, gel, and the like.

Preferably, the ASMT expression promoter, the melatonin synthesis promoter, the antioxidant, or the composition of the present invention comprise ectoin or a physiologically acceptable salt thereof at an amount such that the effect of the promotion of the ASMT expression can be sufficiently exerted.

The amount of ectoin or a physiologically acceptable salt thereof in the ASMT expression promoter, the melatonin synthesis promoter, the antioxidant, or the composition of the present invention can be determined as appropriate depending on the type, purpose, form, method of use, etc. thereof.

When administered externally, the dose of ectoin or a physiologically acceptable salt thereof may be for example, 0.001 to 50% by weight, 0.01 to 5% by weight, 0.01 to 1% by weight, 0.01 to 0.1% by weight, 0.02 to 0.05% by weight, as the amount of ectoin or a physiologically acceptable salt thereof in the total weight of the ASMT expression promoter, the melatonin synthesis promoter, the antioxidant, or the composition of the present invention, although it is not limited thereto.

When administered orally, the amount of ectoin or a physiologically acceptable salt thereof may be for example, 0.001 to 50% by weight, 0.01 to 5% by weight, 0.01 to 1% by weight, 0.01 to 0.1% by weight, 0.02 to 0.05% by weight, in the total weight of the ASMT expression promoter, the melatonin synthesis promoter, the antioxidant, or the composition of the present invention, although it is not limited thereto.

The frequency of administration may be arbitrarily selected from once every four weeks, once every two weeks, once a week, once every three days, once every two days, once a day, twice a day, three times a day, four times a day, five times a day, and each time, etc, although it is not limited thereto.

The ASMT expression promoter, the melatonin synthesis promoter, the antioxidant, or the composition of the present invention may be used together with optionally selected additive(s) as necessary. The additive may include an excipient and the like.

The excipient may be any usual one for use in the desired dosage form, including for example, starches such as wheat starch, rice starch, corn starch, potato starch, dextrin, cyclodextrin, crystalline celluloses, saccharides such as lactose, glucose, sugar, reduced maltose, starch syrup, fructooligosaccharides, emulsified oligosaccharides, sugar alcohols such as sorbitol, erythritol, xylitol, lactitol, mannitol, and the like. These excipients may be used alone or in combination of two or more.

Other additives such as colorants, preservatives, thickeners, binders, disintegrants, dispersants, stabilizers, gelling agents, antioxidants, surfactants, preservatives, pH adjusters, oils, water, alcohols, chelating agents, silicones, ultraviolet absorbers, moisturizers, fragrances, various medicinal ingredients, antiseptic agents, neutralizing agents and such well-known substances may also be appropriately selected and used.

Moreover, the present invention provides a method for promoting melatonin synthesis through the promotion of ASMT expression, by administering ectoine or a physiologically acceptable salt thereof. The present invention also provides a method for enhancing one or more effects selected from improvement of sleep disorder, improvement of cognitive function, improvement of mood disorder, enhancement of antioxidant activity in the skin, and enhancement of anti-inflammatory activity in the skin, by promoting melatonin synthesis through the promotion of ASMT expression, by administering ectoine or a physiologically acceptable salt thereof. The method of the present invention may be a method for cosmetic purposes which is not a treatment to be conducted by a medical practitioner or a medical staff.

Furthermore, the present invention provides the use of ectoine or a physiologically acceptable salt thereof in the manufacture of a medicament for enhancing one or more effects selected from improvement of sleep disorder, improvement of cognitive function, improvement of mood disorder, enhancement of antioxidant activity in the skin, and enhancement of anti-inflammatory activity in the skin. The present invention also provides ectoine or a physiologically acceptable salt thereof for use in a method for enhancing one or more effects selected from improvement of sleep disorder, improvement of cognitive function, improvement of mood disorder, enhancement of antioxidant activity in the skin, and enhancement of anti-inflammatory activity in the skin, by promoting melatonin synthesis through the promotion of ASMT expression.

### EXAMPLES

Next, the present invention will be described in more detail by way of examples.

However, the present invention is not limited thereto.

### Example 1

Effect of Promoting ASMT Gene Expression of Ectoin

Preparation of Sample

Commercially available ectoin (Merck) was dissolved in purified water and filter sterilized to prepare an ectoin sample having the concentration of 1M.

Culture of Epidermal Keratinocytes

Commercially available normal adult skin-derived keratinocytes (Kurabo) were inoculated into a T75 flask at 5×10⁵ cells/flask, and were cultured in a keratinocyte medium (Humedia-KG2, Kurabo) at 37° C. in a 5% CO₂ atmosphere until they reached semiconfluency. The resulting cells were used as seed cells. The keratinocytes were collected by trypsinization, and inoculated into a 24-well plate at 2×10⁴ cells/well, and cultured in a keratinocyte medium (Humedia-KG2, Kurabo) at 37° C., 5% CO₂ atmosphere until they reached confluency.

Addition of Sample

To the epidermal keratinocytes having reached confluency was added the ectoin sample at an appropriate dilution ratio so that the ectoin concentration in the medium was 1 mM, followed by the continuation of the culture at 37° C. in a 5% CO₂ atmosphere. As a control, a medium not containing the ectoin sample was used to carry out the culture.

RNA Extraction from Cells

Ten hours after the addition of ectoin, the medium was removed. Then the cells were lysed and RNA was extracted therefrom using a commercially available RNA extraction reagent (RNeasy Mini Kit, Qiagen).

Evaluation of ASMT Expression Level by Quantitative PCR Method

Using the thus extracted RNA as a template, quantitative PCR was performed with a commercially available quantitative PCR reagent (BrilliantII QRT-PCR kit, Agilent) and a quantitative PCR device (Mx-3005P, Agilent), by which the expression level of the ASMT gene was determined. At the same time, the expression level of the RPLP0 gene whose expression level was almost constant was also determined as an internal standard. Commercially available PCR primer set (Takara Bio Inc.) specific to each gene was used.

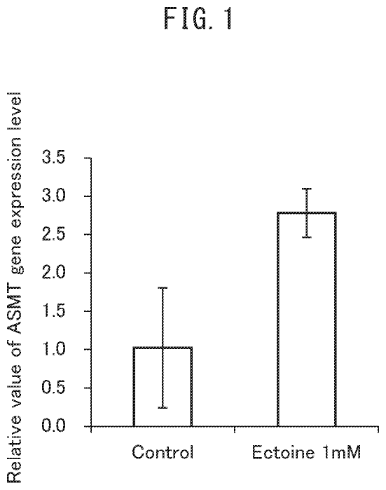

The results are shown in FIG. 1. The ASMT gene expression level obtained from the addition of each sample is shown as a comparison relative to the ASMT gene expression level of the control which is set to 1. From FIG. 1, it was confirmed that ectoin has a very high ASMT expression promoting action as compared with the case where the culture was conducted in the medium containing no ectoin.

### Example 2

Concentration-Dependency of Ectoine for the ASMT Gene Expression

Preparation of Sample

Commercially available ectoin (Merck) was dissolved in purified water and filter sterilized to prepare a 10 wt % ectoin sample.

Addition of Samples

To the epidermal keratinocytes cultured in the same manner as in Example 1 was added the ectoin sample at appropriate dilution ratios so that the ectoin concentration in the medium was 0.005 wt %, 0.01 wt %, 0.02 wt %, 0.05 wt %, 0.1 wt %, followed by the continuation of the culture at 37° C. in a 5% CO₂ atmosphere.

Extraction of RNA

Ten hours after the addition of ectoin, the medium was removed. Then the cells were lysed and RNA was extracted therefrom using a commercially available RNA extraction reagent (RNeasy Mini Kit, Qiagen).

Evaluation of ASMT Expression Level by Quantitative PCR Method

Using the thus extracted RNA as a template, quantitative PCR was performed with a commercially available quantitative PCR reagent (BrilliantII QRT-PCR kit, Agilent) and a quantitative PCR device (Mx-3005P, Agilent), by which the expression level of the ASMT gene was determined. At the same time, the expression level of the RPLP0 gene whose expression level was almost constant was also determined as an internal standard. Commercially available PCR primer set (Takara Bio Inc.) specific to each gene was used.

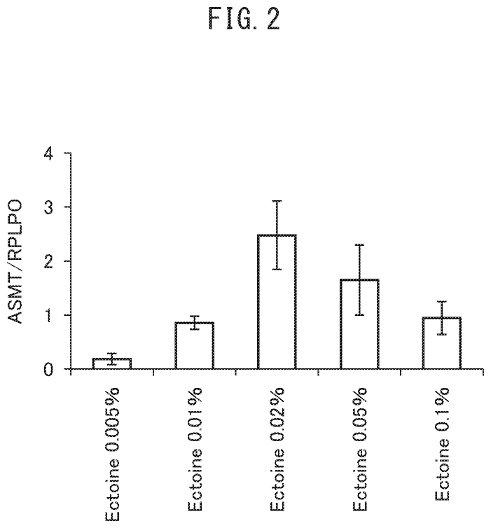

The results are shown in FIG. 2. The ASMT gene expression level obtained from the addition of 0.02 wt % of ectoine was higher than the cases of lower concentrations of ectoine (0.005 wt %, 0.01 wt %). Thus, it was confirmed that the ASMT expression promoting action of ectoin was dependent on the concentration thereof.

### Example 3

ASMT Expression Promoting Action of Ectoin in Comparison with Various Antioxidants

Preparation of Samples

The ectoin sample was prepared in the same manner as in Example 1. As control drugs, samples respectively containing substances known to have an antioxidant action (glycosyl hesperidin, green tea extract, oolong tea extract) were also prepared. A commercially available glycosyl hesperidin (Toyo Sugar) was dissolved in purified water and filter sterilized to prepare a 100 mM glycosyl hesperidin sample. Commercially available green tea extract (Koei Kogyo) and oolong tea extract (Suntory) were filter sterilized.

To the epidermal keratinocytes cultured in the same manner as in Example 1 was added the respective samples at appropriate dilution ratios so that the concentration in the medium was respectively 1 mM (ectoine), 100 μM (glycosyl hesperidin), 0.1% (green tea extract), and 0.1% (oolong tea extract). These concentrations are all normal concentrations to be used in external formulations. After the addition of the samples, the cells were cultured, from which the ASMT expression level was evaluated in the same manner as in Example 1. As a control, a medium not containing any sample was used to carry out the culture.

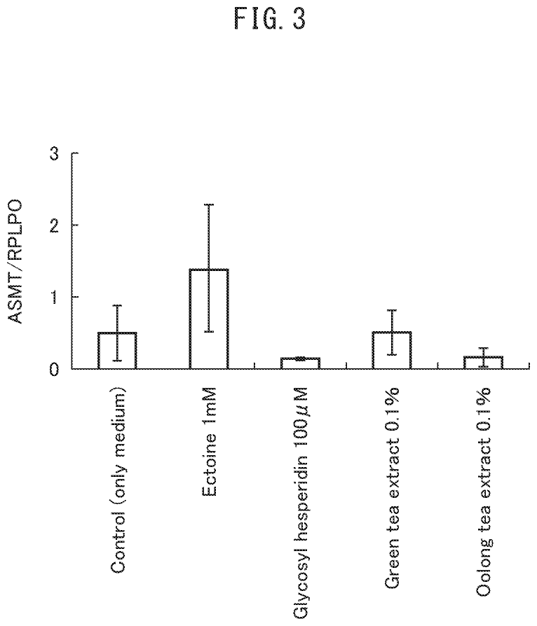

The results are shown in FIG. 3. From FIG. 3, it was confirmed that the ASMT gene expression level of ectoin was higher than that of green tea extract, oolong tea extract, and glycosyl hesperidin at even higher concentration.

### INDUSTRIAL AVAILABILITY

The present invention is capable of promoting the expression of ASMT gene by the administration of the ASMT expression promoter comprising ectoin or a physiologically acceptable salt thereof, as well as enhancing one or more effects selected from sleep disorder improvement, cognitive function enhancement, mood disorder improvement, anti-oxidant activity enhancement, and anti-inflammatory activity enhancement, by promoting the synthesis of melatonin through promoting the expression of ASMT gene.

1. An ASMT expression promoter comprising ectoine or a physiologically acceptable salt thereof as an active ingredient.

2. A melatonin synthesis promoter comprising ectoine or a physiologically acceptable salt thereof as an active ingredient, which promotes melatonin synthesis through the promotion of ASMT expression.

3. An antioxidant comprising ectoine or a physiologically acceptable salt thereof as an active ingredient, which enhances antioxidant activity by promoting melatonin synthesis through the promotion of ASMT expression.

4. A composition comprising the ASMT expression promoter according to claim 1.

5. A composition comprising the melatonin synthesis promoter according to claim 2.

6. A composition comprising the antioxidant according to claim 3.

7. The composition according to claim 4 for enhancing one or more effects selected from improvement of sleep disorder, improvement of cognitive function, improvement of mood disorder, enhancement of antioxidant activity, and enhancement of anti-inflammatory activity, by promoting melatonin synthesis through the promotion of ASMT expression.

8. The composition according to claim 4, which is a cosmetic composition or a food composition.

9. The composition according to claim 5 for enhancing one or more effects selected from improvement of sleep disorder, improvement of cognitive function, improvement of mood disorder, enhancement of antioxidant activity, and enhancement of anti-inflammatory activity, by promoting melatonin synthesis through the promotion of ASMT expression.

10. The composition according to claim 5, which is a cosmetic composition or a food composition.

11. The composition according to claim 6, which is a cosmetic composition or a food composition.

12. The composition according to claim 7, which is a cosmetic composition or a food composition.

13. The composition according to claim 9, which is a cosmetic composition or a food composition.
